(19) United States
(12) Patent Application Publication
(10) Pub. No.: US 2024/0207277 A1
(43) Pub. Date: Jun. 27, 2024

(54) Methods For Treating Drug and Vaccine Induced Immune Thrombocytopenia by Administering Specific Compounds

(71) Applicant: Principia Biopharma Inc., Bridgewater, NJ (US)

(72) Inventors: Christopher W. Smith, Coventry (GB); Steve P. Watson, Droitwich Worcestershire (GB); Phillip L.R. Nicolson, Birmingham (GB); Claire Langrish, Redwood City, CA (US)

(73) Assignee: Principia Biopharma Inc., Bridgewater, NJ (US)

(21) Appl. No.: 18/555,388

(22) PCT Filed: Apr. 14, 2022
(86) PCT No.: PCT/US2022/024806

§ 371 Date: Oct. 13, 2023

Related U.S. Application Data
(60) Provisional application No. 63/175,976, filed on Apr. 16, 2021.

(51) Int. Cl. A61K 31/519; A61P 7/02; A61P 7/04

(52) U.S. Cl. CPC A61K 31/519; A61P 7/02; A61P 7/04

(57) ABSTRACT

Methods for treating and/or preventing drug-induced thrombocytopenia (DITP) and vaccine-induced thrombosis and thrombocytopenia syndrome (VITT) with certain BTK inhibitors and/or pharmaceutically acceptable salts thereof are provided.

### BACKGROUND AND SUMMARY

Drug-induced thrombocytopenia (DITP) and vaccine-induced thrombosis and thrombocytopenia (VITT) have a clinical resemblance to autoimmune heparin induced thrombocytopenia (HIT), with the majority of patients testing positive for anti-platelet factor 4 (PF4) antibodies. Greinacher et al., Thrombotic thrombocytopenia after ChAdox1 nCov-19 vaccination, N Engl J Med, doi:10.1056/NEJMoa2104840 (2021); Greinacher et al., Autoimmune heparin-induced thrombocytopenia, J Thromb Haemost 15(11):2099-114 (2017). In HIT, IgG-containing immune complexes bind and cross-link the platelet surface receptor FcγRIIA (CD32a), a low affinity Fc receptor (FcR) that binds immune complexes with high avidity, and initiate platelet activation. Greinacher et al., Autoimmune heparin-induced thrombocytopenia, J Thromb Haemost 15(11):2099-114 (2017). Autoimmune HIT, despite the name, is rare but occurs independently of heparin and result in persistent severe thrombocytopenia together with DIC and microvascular thrombosis. Id.

Novel, safe, and effective oral treatments to maintain platelet counts in DITP and VITT patients would represent a significant therapeutic advantage over current standard of care. Accordingly, disclosed herein are novel methods for the treatment and/or prevention of DITP and VITT with specific compounds.

Bruton's agammaglobulinemia tyrosine kinase (BTK) is an essential signaling element downstream of the B-cell receptor (BCR), Fc-gamma receptor (FcγR), and Fc-epsilon receptor (FcR). BTK is a non-receptor tyrosine kinase and a member of the TEC family of kinases. BTK is essential to B cell lineage maturation, and inhibition of BTK activity in cells produces phenotypic changes consistent with blockade of the BCR. Illustratively, BTK inhibition results in the down-regulation of various B-cell activities, including cell proliferation, differentiation, maturation, and survival, and the up-regulation of apoptosis.

Rather than acting in an “on/off switch” manner, BTK may be best viewed as an immune function “modulator” (Crofford U et al., 2016; Pal Singh S et al., 2018). Important insights into BTK function come from loss of function analyses in humans and mice. Individuals with loss of function mutations in the BTK gene develop X-linked agammaglobulinemia (XLA), characterized by a complete absence of circulating B cells and plasma cells, and very low levels of immunoglobulins of all classes (Tsukada 1993, Vetrie 1993). This indicates the potential for BTK inhibition to suppress production of autoantibodies thought to be important in the development of autoimmune diseases.

While BTK is not expressed in T cells, natural killer cells, and plasma cells and has no traceable direct functions in T cells and plasma cells (Sideras and Smith 1995; Mohamed et al., 2009), the enzyme regulates the activation of other hematopoietic cells, such as basophils, mast cells, macrophages, neutrophils, and platelets. For example, BTK plays a role in the activation of neutrophils, which are key players in the inflammatory response that contributes to wound healing but may also cause tissue damage (Volmering S et al., 2016).

Accordingly, a selective BTK inhibitor has the potential to target multiple pathways involved in inflammation and autoimmunity, including, but not limited to: blocking BCR; inhibiting plasma cell differentiation and antibody production; blocking IgG-mediated FcγR activation, phagocytosis, and inflammatory mediators in monocytes or macrophages; blocking IgE-mediated FcεR activation and degranulation in mast cells or basophils; and inhibiting activation, adhesion, recruitment, and oxidative burst in neutrophils. Based on these effects, a selective BTK inhibitor may block the initiation and progression of various inflammatory diseases and mitigate tissue damage resulting from these diseases. Although individuals with loss of function mutations in the BTK gene have decreased humoral immunity and are susceptible to pyogenic bacterial and enterovirus infections, requiring treatment with intravenous immunoglobulin, inhibition of BTK in individuals with an intact immune system is not predicted to produce similar susceptibility to infection.

Several orally administered BTK inhibitors (BTKi), including ibrutinib (PCI-32765) and spebrutinib (CC-292), are currently marketed or in clinical development for a range of indications (Lee A et al., 2017). For example, ibrutinib has provided further clinical validation of the BTK target and was recently approved for human use in mantle cell lymphoma, Waldenström's macroglobulinemia, and chronic lymphocytic leukemia by the U.S. Food and Drug Administration (FDA) (Imbruvica Package Insert, 2015). Ibrutinib has also demonstrated activity in other hematological malignancies (Wang 2013 Byrd 2013),), and most recently for use as an antiplatelet agent (Nicolson P L et al. (2020) Haematologica 106(1):208-219; doi.org/10.3324/haematol.2019.218545) and for counteracting thrombocytopenia occurring shortly after vaccination with the COVID-19 vaccine AZD1222 (Vaxzevria) (von Hundelshausen et al. (2021) Thromb Haemost. April 13. Doi 10.1055/a-1481-3039). In addition, CC-292 has been reported to be well tolerated in a healthy volunteer population at doses which provide 100% occupancy of the BTK enzyme (Evans 2013). Furthermore, evobrutinib recently demonstrated efficacy for multiple sclerosis in a Phase 2 trial (Montalban X et al., 2019). Other BTKi compounds are in clinical development for various immune-mediated disorders, such as pemphigus (NCT02704429), rheumatoid arthritis (NCT03823378, NCT03682705, NCT03233230), and asthma (NCT03944707) (Montalban X et al., 2019; Norman P 2016; Tam C S et al., 2018; Crawford J J et al., 2018; Min T K et al., 2019; Gillooly K M 2017; Nadeem A et al., 2019).

Some BTK inhibitors can cause bleeding and therefore would not be ideal candidates for use in subjects who are thrombocytopenic, anticoagulated, and/or have intracerebral bleeding. Langrish C L et al. (2021) J Immunol. April 1; 206(7):1454-1468. PMID: 33674445; PMCID: PMC7980532. However, Compound (I) as disclosed has no impact on normal platelet function in vitro and has not been associated with bleeding in thrombocytopenic patients, and in fact is being investigated in treating immune thrombocytopenia (ITP).

Compound (I), also known as “rilzabrutinib,” as described herein, is a BTK inhibitor of the following structure:

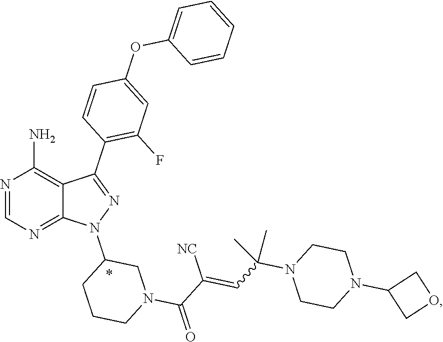 [SMILES: CC(C)(C=C(C#N)C(=O)N1CCCC(n2nc(-c3ccc(Oc4ccccc4)cc3F)c3c(N)ncnc32)C1)N1CCN(C2COC2)CC1]

wherein *C is a stereochemical center. See PCT Publication No. WO 2014/039899, which is incorporated herein by reference, e.g., Example 31.

This compound has been disclosed in several patent publications, such as, e.g., PCT Publication Nos. WO 2014/039899, WO 2015/127310, WO 2016/100914, WO 2016/105531, and WO 2018/005849, the contents of each of which are incorporated by reference herein.

Rilzabrutinib is a novel, highly selective, small molecule inhibitor of non-T cell white blood cell signaling via B-cell receptor, FCγR, and/or FcεR signaling of the BTK pathway. Rilzabrutinib functions as a reversible covalent BTK inhibitor and forms both a non-covalent and a covalent bond with its target, allowing for enhanced selectivity and extended inhibition with low systemic exposure. In comparison to first and second generation BTKi, rilzabrutinib has shown minimal cross-reactivity with other molecules and is low risk for off-target effects (Smith P F et al., 2017). Importantly, rilzabrutinib's reversible binding minimizes the likelihood of permanently modified peptides (Serafimova IM 2012). In addition, rilzabrutinib shows improved kinase selectivity relative to the covalent BTK inhibitor ibrutinib, with rilzabrutinib (1 μM) achieving >90% inhibition for 6 kinases compared to 21 kinases for ibrutinib (1 μM) in a 251-kinase panel.

Rilzabrutinib has shown encouraging results for the treatment of immune-mediated diseases. Rilzabrutinib is the most advanced BTKi in development for an autoimmune disease (Phase 3, NCT03762265) and the first BTKi to be evaluated in the treatment of pemphigus, a blistering disease that, like ITP, is autoantibody-driven. In humans, rilzabrutinib is rapidly absorbed following oral administration, with a fast half-life (3-4 h) and variable pharmacokinetics (PK).

Compound (II), also known as “atuzabrutinib,” as described herein is a BTK inhibitor of the following structure:

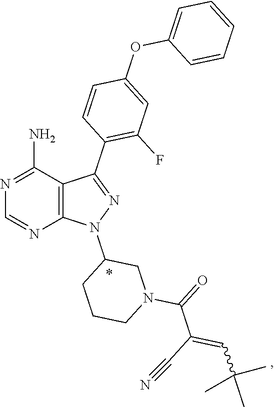 [SMILES: CC(C)(C)C=C(C#N)C(=O)N1CCCC(n2nc(-c3ccc(Oc4ccccc4)cc3F)c3c(N)ncnc32)C1]

where *C is a stereochemical center. This compound has been disclosed in e.g., WO 2012/158764 (see, e.g., Compound 125A/125B in Table 1), which is incorporated herein by reference.

In accordance herein, the following non-limiting embodiments are encompassed:
Embodiment 1. A method for treating or preventing drug-induced thrombocytopenia (DITP) in a human subject in need thereof comprising administering to the human subject a therapeutically effective amount of at least one BTK inhibitor selected from Compound (I), Compound (II), and pharmaceutically acceptable salts thereof.
Embodiment 2. A method for treating or preventing vaccine-induced thrombosis and thrombocytopenia syndrome (VITT) in a human subject in need thereof comprising administering to the human subject a therapeutically effective amount of at least one BTK inhibitor selected from Compound (I), Compound (II), and pharmaceutically acceptable salts thereof.
Embodiment 3. A method for increasing platelet count in a human subject with drug-induced thrombocytopenia (DITP) or vaccine-induced thrombosis and thrombocytopenia syndrome (VITT) comprising administering to the human subject a therapeutically effective amount of at least one BTK inhibitor selected from Compound (I), Compound (II), and pharmaceutically acceptable salts thereof.
Embodiment 4. A method for reducing platelet aggregation in a human subject with drug-induced thrombocytopenia (DITP) or vaccine-induced thrombosis and thrombocytopenia syndrome (VITT) comprising administering to the human subject a therapeutically effective amount of at least one BTK inhibitor selected from Compound (I), Compound (II), and pharmaceutically acceptable salts thereof.
Embodiment 5. The method of any one of embodiments 1-4, wherein, prior to administration, the human subject has at least one characteristic chosen from:
   a. elevated D-dimer levels;
   b. thrombosis; and
   c. anti-platelet factor 4 (PF4) antibodies.
Embodiment 6. The method of any one of embodiments 1, and 3-5, wherein the drug-induced thrombocytopenia (DITP) is induced by administration of a small molecule, protein, or components, diluents, excipients or the like found in therapeutic treatments.
Embodiment 7. The method of any one of embodiments 1, and 3-6, wherein the drug-induced thrombocytopenia (DITP) is induced by administration of unfractionated heparin, enoxaparin, dalteparin, tinzaparin, acenocoumarol, acetaminophen, acetyldigoxin, alfacalcidol, allopurinol, alteplase, amphotericin B, argatroban, aspirin, atenolol, azathioprine, bivalirudin, bortezomib, capecitabine, captopril, carbamazepine, carboplatin, carfilzomib, ceftriaxone, cephalexin, chlorthalidone, cilastin/imipenem, clopidogrel, clozapine, cyclocytidine, dactinomucin/actinomycin, deferasirox, deferiprone, diflunisal, digoxin, dipyridamole, drospirenone/ethinylestradiol, eltrombopag, epoetin alfa, eporestenol, eptifibatide, famotidine, fluconazole, fluorouracil, furosemide, fusidic acid, ganciclovir, gemcitabine, gentamicin, glycoprotein IIB/IIA inhibitor, gold, hydrochlorothiazide, hydrochlorothiazide/triamterene, hydroxychloroquine, imatinib, inamrinone, intergrilin, ITP drugs, ixazomib, lenalidomide, levetiracetam, linezolid, melperone, menatetrenone, meropenem, methotrexate, metoprolol, molsidomine, nedaplatin, nicotinamide, nitrofurantoin, nonsteroidal anti-inflammatory drugs (NSAIDS) (e.g., ibuprofen, naproxen, celecoxib, diclofenac, and the like), octreotide, pantroprazole, penicillamine, phenytoin, piperacillin, propranolol, proton pump inhibitors, quinine, rifampin, ruxolitinib, ruxolitnib phosphate, sirolimus, spironolactone, streptokinase, sulfamethoxazole, sulfisoxazole, sunitinib, teicoplanin, temozolomide, temsirolimus, ticlopidine, tirofiban, trimethoprim/sulfamethoxazole, urokinase, valganciclovir, valproic acid, vancomycin, warfarin, or combinations thereof.
Embodiment 8. The method of any one of embodiments 1, and 3-6, wherein the drug-induced thrombocytopenia (DITP) is induced by administration of filgrastim (granulocyte colony stimulating factor; G-CSF), interferon, interferon alpha, peginterferon alfa 2B, peginterferon alfa 2B/ribavirin, factor VIII, TNF alpha, INF gamma, or combinations thereof.
Embodiment 9. The method of any one of embodiments 1, and 3-6, wherein the drug-induced thrombocytopenia (DITP) is induced by administration of abciximab, adalimumab, alemtuzumab, antibody-drug conjugates, anti-thymocyte globulin, brentuximab, cixutumumab, efaluzumab, natalizumab, rituximab, trastuzumab, or combinations thereof.
Embodiment 10. The method of any one of embodiments 2-5, wherein the vaccine-induced thrombosis and thrombocytopenia syndrome (VITT) is induced by administration of a vaccine.
Embodiment 11. The method of any one of embodiments 2-5, and 10, wherein the vaccine-induced thrombosis and thrombocytopenia syndrome (VITT) is induced by administration of a vaccine delivered in an adenoviral vector.
Embodiment 12. The method of embodiment 11, wherein the adenoviral vector comprises a therapeutic and/or prophylactic agent.
Embodiment 13. The method of embodiment 12, wherein the therapeutic or prophylactic agent is a gene therapy.
Embodiment 14. The method of any one of embodiments 10-13, wherein the vaccine is to prevent a coronavirus infection.
Embodiment 15. The method of embodiment 14, wherein the coronavirus infection is COVID-19.
Embodiment 16. The method of any one of embodiments 10-13, wherein the vaccine is to prevent an infection selected from measles, mumps, rubella, varicella, herpes simplex virus 1, herpes simplex virus 2, varicella, rotavirus, influenza, yellow fever, smallpox, hepatitis B, human papilloma virus, pneumococcus, hepatitis A, anthrax, diphtheria, acellular pertussis, hemophilus influenzae, including type B, meningococcus C, meningitis, typhoid, rabies, Lyme disease, tetanus, or any combination thereof.
Embodiment 17. The method of any one embodiments 1-16, wherein Compound I is (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, (S)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, a mixture of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and (S)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile; or an individual (E)- or (Z)-isomer of any of the above compounds; and/or a pharmaceutically acceptable salt of any of the above compounds.
Embodiment 18. The method of embodiment 17, wherein Compound I is the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt thereof.
Embodiment 19. The method of embodiment 17, wherein Compound I is the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt thereof.
Embodiment 20. The method of embodiment 17, wherein Compound I is a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt thereof.
Embodiment 21. The method of any one of embodiments 1-17, wherein Compound II is (R)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile, (S)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile, a mixture of (R)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile and (S)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile, or an individual (E)- or (Z)-isomer of any of the above compounds; and/or a pharmaceutically acceptable salt of any of the above compounds.
Embodiment 22. The method of embodiment 21, wherein Compound II is the (E) isomer of (R)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile or a pharmaceutically acceptable salt thereof.
Embodiment 23. The method of embodiment 21, wherein Compound II is the (Z) isomer of (R)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile or a pharmaceutically acceptable salt thereof.
Embodiment 24. The method of embodiment 21, wherein Compound II is a mixture of (E) and (Z) isomers of (R)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile or a pharmaceutically acceptable salt thereof.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIGS. 1A-1C show serum from patients with vaccine-induced thrombosis and thrombocytopenia syndrome (VITT) induces platelet aggregation via the FcγRIIA. Washed platelets (2×10⁸/mL) were stimulated with serum (1:15, v/v) from healthy donors (HD) or patient with VITT (P) pre- and post-intravenous immunoglobulin (IVIg) treatment or in the presence of 10 μg/mL IV.3 F(ab), low concentration heparin (0.2 U/mL), or following heat inactivation (56° C., 45 minutes). Platelet aggregation was measured for each condition. FIG. 1A shows representative aggregation traces. FIGS. 1B and 1C depict the quantification of maximum aggregation through area under the curve (AUC), as measured for 10 minutes for P2, P3, P4, and P7 pre- and post-IVIg samples (FIG. 1B), and P1, P5, and P6 post-IVIg (FIG. 1C) and plasma exchange samples. Mean±SEM, n=3. Statistical analysis was by two-way ANOVA with Dunnett multiple comparisons, * p<0.05, ns: non-significant.

FIGS. 2A-B show the effect of Btk inhibition by rilzabrutinib, which blocks platelet aggregation induced by serum from patients with VITT. Washed platelets (2×10⁸/mL) were incubated with rilzabrutinib (0.5 μM) or vehicle (0.02% DMSO) for 10 minutes then stimulated with serum (1:15, v/v) from patients with VITT. FIG. 2A shows representative aggregation traces. FIG. 2B shows quantification of maximum aggregation. Statistical analysis was by one-way ANOVA with Dunnett multiple comparisons. Mean±SEM, n=7·*p<0.05.

### DETAILED DESCRIPTION

### Definitions

Unless otherwise stated, the following terms used in the specification and claims are defined for the purposes of this Application and have the following meanings. All undefined technical and scientific terms used in this Application have the meaning as commonly understood by one of ordinary skill in the art to which this disclosure belongs.

As used herein, “a” or “an” entity refers to one or more of that entity; for example, a compound refers to one or more compounds or at least one compound unless stated otherwise. As such, the terms “a” (or “an”), “one or more”, and “at least one” can be used interchangeably herein.

As used herein, the term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 5%. With regard to specific values, it should be understood that specific values described herein for subject populations (e.g., the subject of the described clinical trial) represent median, mean, or statistical numbers, unless otherwise provided. Accordingly, aspects of the present disclosure requiring a particular value in a subject are supported herein by population data in which the relevant value is assessed to be a meaningful delimitation on the subject population.

As used herein, the term “active pharmaceutical ingredient” or “therapeutic agent” (“API”) refers to a biologically active compound.

As used herein, the terms “administer,” “administering,” or “administration” herein refer to providing, giving, dosing, and/or prescribing by either a health practitioner or an authorized agent and/or putting into, taking or consuming by the patient or person himself or herself. For example, “administration” of an API to a patient refers to any route (e.g., oral delivery) of introducing or delivering the API to the patient. Administration includes self-administration and administration by another.

As used herein, “immune thrombocytopenia” (ITP) encompasses or at least also refers to other terms commonly used such as idiopathic thrombocytopenia and idiopathic thrombocytopenic purpura. There are two main types of ITP: short (acute) and chronic (long term). Acute ITP typically lasts less than six months, whereas chronic ITP can last six months or longer. ITP affects multiple age groups and can be seen in children, teenagers, and adults.

ITP is a disorder that can lead to easy or excessive bruising and bleeding. The bleeding results from unusually low levels of platelets. ITP may result from the development of an antibody directed against a structural platelet antigen. In childhood ITP, the antibody may be triggered by viral antigens. In adults, the trigger is unknown, although ITP has been associated with Helicobacter pylori infections, and treatment of the infections has been followed by remission of the ITP. ITP may worsen during pregnancy and may increase the risk of maternal morbidity. In some embodiments, ITP may be induced by a drug (i.e., drug-induced immune thrombocytopenia; DITP), such as a small molecule or an antibody.

As used herein, “drug-induced immune thrombocytopenia” (DITP) refers to acute, immune-mediated thrombocytopenia, which may be suspected when a subject has sudden, severe thrombocytopenia. DITP is induced by a drug. Exemplary and non-limiting drugs that can induce DITP include small molecules, proteins, antibodies, as well as compositions and/or compounds used in therapeutic treatments.

As used herein, “vaccine-induced immune thrombosis and thrombocytopenia” (VITT) refers to blood clotting with low levels of platelets (i.e., thrombosis with thrombocytopenia), disseminated intravascular coagulation (DIC) and bleeding with high mortality that is induced by a vaccine. In some embodiments, VITT is induced by a COVID-19 vaccine. COVID-19 vaccines may comprise whole virus, attenuated virus, viral particle, protein, nucleic acid, and/or viral vector. In some embodiments, the COVID-19 vaccine is a viral vector vaccine. In some embodiments, the COVID-19 vaccine is an adenoviral vector vaccine. In some embodiments, the vaccine is AZD1222 (Oxford-AstraZeneca; formerly ChAdox1 nCoV-19). VITT may sometimes be called “vaccine-induced prothrombotic immune thrombocytopenia (VIPIT),” and both are encompassed herein (i.e., VITT includes VITT and VIPIT).

As used herein, a “pharmaceutically acceptable carrier or excipient” means a carrier or an excipient that is useful in preparing a pharmaceutical composition that is generally safe, and neither biologically nor otherwise undesirable, such as, e.g., a carrier or an excipient that is acceptable for mammalian pharmaceutical use.

As used herein, the term “pharmaceutically acceptable salt” refers to a salt form, e.g., an acid addition salt, of an active pharmaceutical agent that is pharmaceutically acceptable and that possesses the desired pharmacological activity of the API of which the salt is made. Pharmaceutically acceptable salts are well known in the art and include those derived from suitable inorganic and organic acids. Such salts include, but are not limited to, salts formed with inorganic acids such as hydrochloric acid, hydrobromic acid, sulfuric acid, phosphoric acid, and the like; or formed with organic acids such as formic acid, acetic acid, propionic acid, hexanoic acid, lactic acid, malonic acid, succinic acid, malic acid, maleic acid, fumaric acid, tartaric acid, citric acid, benzoic acid, mandelic acid, methanesulfonic acid, ethanesulfonic acid, 1,2-ethanedisulfonic acid, benzenesulfonic acid, 4-toluenesulfonic acid, and the like. S. M. Berge et al. describes pharmaceutically acceptable salts in detail in J. Pharmaceutical Sciences, 1977, 66, 1-19.

As used herein, the terms “Compound (I),” “rilzabrutinib,” “(R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile” and “2-[(3R)-3-[4-amino-3-(2-fluoro-4-phenoxy-phenyl)pyrazolo[3,4-d]-pyrimidin-1-yl]piperidine-1-carbonyl]-4-methyl-4-[4-(oxetan-3-yl)piperazin-1-yl]pent-2-enenitrile” are used interchangeably to refer to a compound having the structure:

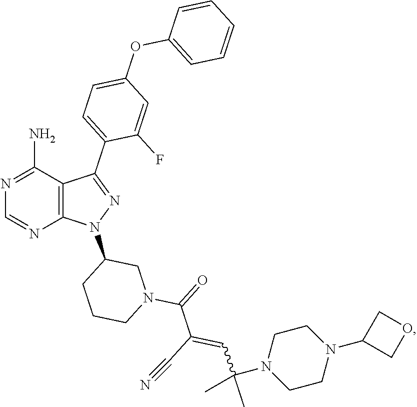 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1]

where *C is a stereochemical center.

A dose of rilzabrutinib may contain the corresponding (S) enantiomer as an impurity in less than about 5% by weight, such as, e.g., as an impurity in less than about 1% by weight. Similarly, a dose of the (E) isomer of rilzabrutinib may contain the corresponding (Z) isomer as an impurity in less than about 1% by weight; a dose of the (Z) isomer of rilzabrutinib may contain the corresponding (E) isomer as an impurity in less than about 1% by weight. When rilzabrutinib is denoted as a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, it means that the amount of (E) or (Z) isomer in the mixture is greater than about 1% by weight. In some embodiments, the molar ratio of (E) to (Z) isomer is 9:1.

As used herein, “Compound (II)” and “atuzabrutinib,” are used interchangeably to refer to the (E) isomer, (Z) isomer, or a mixture of (E) and (Z) isomers of (R)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile, (S)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile, or a mixture of (R) and (S) enantiomers of 2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile, which has the following structure:

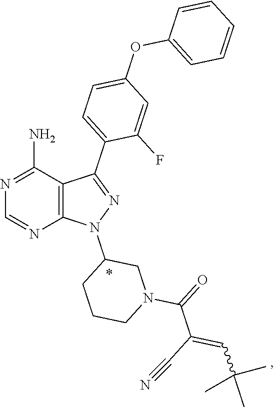 [SMILES: CC(C)(C)C=C(C#N)C(=O)N1CCCC(n2nc(-c3ccc(Oc4ccccc4)cc3F)c3c(N)ncnc32)C1]

where *C is a stereochemical center.

When Compound (II) is denoted as (R)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile, it may also contain the corresponding (S) enantiomer as an impurity in less than 5% by weight, such as, e.g., an impurity in less than 1% by weight. Accordingly, when the Compound (II) is denoted as a mixture of (R) and (S) enantiomers of 2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile, the amount of (R) or (S) enantiomer in the mixture is greater than 1% by weight. Similarly, when Compound (II) is denoted as the (E) isomer, it may contain the corresponding (Z) isomer as an impurity in less than 5% by weight, such as less than 1% by weight. Accordingly, when the Compound (II) is denoted as a mixture of (E) and (Z) isomers of 2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile, the amount of (E) or (Z) isomer in the mixture is greater than 1% by weight.

In some embodiments, Compound (II) is a mixture of (R) and (S) enantiomers of 2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile.

In some embodiments, Compound (II) is substantially (R)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile. In some embodiments, Compound (II) is at least about 75%, e.g., at least about 80%, at least about 85%, at least about 90%, at least about 95%, by weight (R)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile. In some embodiments, Compound (II) is at least about 95% by weight (R)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile.

As used herein, the term “therapeutically effective amount” refers to that an of a compound that produces the desired effect for which it is administered (e.g., improvement in DITP or a symptom of DITP, or lessening the severity of DITP or a symptom of DITP, or improvement in VITT or a symptom of VITT, or lessening the severity of VITT or a symptom of VITT). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the term “treat,” “treating,” or “treatment,” when used in connection with a disorder or condition, includes any effect, e.g., lessening, reducing, modulating, ameliorating, or eliminating, that results in the improvement of the disorder or condition. Improvements in or lessening the severity of any symptom of the disorder or condition can be readily assessed according to standard methods and techniques known in the art.

“Preventing,” as used herein, includes providing prophylaxis with respect to the occurrence or recurrence of a disease, disorder, or condition in a subject that may be predisposed to the disease, disorder, or condition but has not yet been diagnosed with the disease, disorder, or condition. Unless otherwise specified, the terms “prevent,” “prevention,” “reduce,” “inhibit,” or “prevent” do not denote or require complete prevention over all time.

In accordance with the description, provided herein is a method for treating or preventing drug-induced thrombocytopenia (DITP) in a human subject in need thereof comprising administering to the human subject a therapeutically effective amount of at least one BTK inhibitor selected from Compound (I):

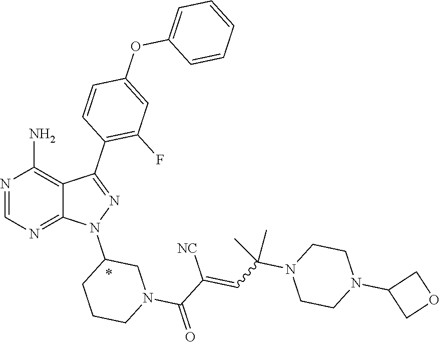 [SMILES: CC(C)(C=C(C#N)C(=O)N1CCCC(n2nc(-c3ccc(Oc4ccccc4)cc3F)c3c(N)ncnc32)C1)N1CCN(C2COC2)CC1]

where *C is a stereochemical center,

### Compound (II):

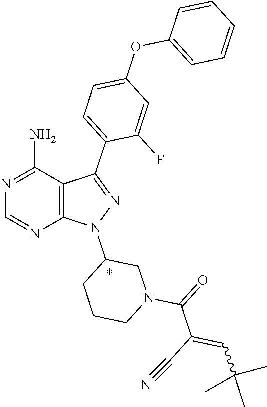 [SMILES: CC(C)(C)C=C(C#N)C(=O)N1CCCC(n2nc(-c3ccc(Oc4ccccc4)cc3F)c3c(N)ncnc32)C1]

where *C is a stereochemical center, and pharmaceutically acceptable salts thereof.

Also provided herein is a method for treating or preventing vaccine-induced thrombosis and thrombocytopenia syndrome (VITT) in a human subject in need thereof comprising administering to the human subject a therapeutically effective amount of at least one BTK inhibitor selected from Compound (I), Compound (II), and pharmaceutically acceptable salts thereof.

In some embodiments, a method for increasing platelet count in a human subject with drug-induced thrombocytopenia (DITP) or vaccine-induced thrombosis and thrombocytopenia syndrome (VITT) comprising administering to the human subject a therapeutically effective amount of at least one BTK inhibitor selected from Compound (I), Compound (II), and pharmaceutically acceptable salts thereof.

In some embodiments, a method for reducing platelet aggregation in a human subject with drug-induced thrombocytopenia (DITP) or vaccine-induced thrombosis and thrombocytopenia syndrome (VITT) comprising administering to the human subject a therapeutically effective amount of at least one BTK inhibitor selected from Compound (I), Compound (II), and pharmaceutically acceptable salts thereof.

In each instance, in some embodiments, prior to administration, the human subject has at least one characteristic chosen from: elevated D-dimer levels, thrombosis; and anti-platelet factor 4 (PF4) antibodies.

In embodiments related to drug-induced thrombocytopenia (DITP), the DITP may be induced by administration of a small molecule, protein, or components, diluents, excipients or the like found in therapeutic treatments.

In some embodiments, the drug-induced thrombocytopenia (DITP) is induced by administration of unfractionated heparin, enoxaparin, dalteparin, tinzaparin, acenocoumarol, acetaminophen, acetyldigoxin, alfacalcidol, allopurinol, alteplase, amphotericin B, argatroban, aspirin, atenolol, azathioprine, bivalirudin, bortezomib, capecitabine, captopril, carbamazepine, carboplatin, carfilzomib, ceftriaxone, cephalexin, chlorthalidone, cilastin/imipenem, clopidogrel, clozapine, cyclocytidine, dactinomucin/actinomycin, deferasirox, deferiprone, diflunisal, digoxin, dipyridamole, drospirenone/ethinylestradiol, eltrombopag, epoetin alfa, eporestenol, eptifibatide, famotidine, fluconazole, fluorouracil, furosemide, fusidic acid, ganciclovir, gemcitabine, gentamicin, glycoprotein JIB/IIA inhibitor, gold, hydrochlorothiazide, hydrochlorothiazide/triamterene, hydroxychloroquine, imatinib, inamrinone, intergrilin, ITP drugs, ixazomib, lenalidomide, levetiracetam, linezolid, melperone, menatetrenone, meropenem, methotrexate, metoprolol, molsidomine, nedaplatin, nicotinamide, nitrofurantoin, nonsteroidal anti-inflammatory drugs (NSAIDS) (e.g., ibuprofen, naproxen, celecoxib, diclofenac, and the like), octreotide, pantroprazole, penicillamine, phenytoin, piperacillin, propranolol, proton pump inhibitors, quinine, rifampin, ruxolitinib, ruxolitnib phosphate, sirolimus, spironolactone, streptokinase, sulfamethoxazole, sulfisoxazole, sunitinib, teicoplanin, temozolomide, temsirolimus, ticlopidine, tirofiban, trimethoprim/sulfamethoxazole, urokinase, valganciclovir, valproic acid, vancomycin, warfarin, or combinations thereof.

In some embodiments, the drug-induced thrombocytopenia (DITP) is induced by administration of filgrastim (granulocyte colony stimulating factor; G-CSF), interferon, interferon alpha, peginterferon alfa 2B, peginterferon alfa 2B/ribavirin, factor VIII, TNF alpha, INF gamma, or combinations thereof.

In some embodiments, the drug-induced thrombocytopenia (DITP) is induced by administration of abciximab, adalimumab, alemtuzumab, antibody-drug conjugates, anti-thymocyte globulin, brentuximab, cixutumumab, efaluzumab, natalizumab, rituximab, trastuzumab, or combinations thereof.

In embodiments relating to vaccine-induced thrombosis and thrombocytopenia syndrome (VITT), the VITT may be induced by administration of a vaccine. In some embodiments, the (VITT) is induced by administration of a vaccine delivered in an adenoviral vector. In certain aspects, the adenoviral vector comprises a therapeutic and/or prophylactic agent. In some embodiments, the therapeutic or prophylactic agent is a gene therapy. In some embodiments, the vaccine is to prevent a coronavirus infection. In some embodiments, the coronavirus infection is COVID-19. In some embodiments, the vaccine is AZD1222 (Oxford-AstraZeneca COVID-19 vaccine).

In some embodiments, the vaccine is to prevent an infection selected from measles, mumps, rubella, varicella, herpes simplex virus 1, herpes simplex virus 2, varicella, rotavirus, influenza, yellow fever, smallpox, hepatitis B, human papilloma virus, pneumococcus, hepatitis A, anthrax, diphtheria, acellular pertussis, hemophilus influenzae, including type B, meningococcus C, meningitis, typhoid, rabies, Lyme disease, tetanus, or any combination thereof.

In some embodiments, Compound I is (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, (S)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, a mixture of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and (S)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile; or an individual (E)- or (Z)-isomer of any of the above compounds; and/or a pharmaceutically acceptable salt of any of the above compounds.

In some embodiments, Compound I is the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt thereof.

In some embodiments, Compound I is the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt thereof.

In some embodiments, Compound I is a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt thereof.

In some embodiments, Compound II is (R)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile, (S)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile, a mixture of (R)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile and (S)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile, or an individual (E)- or (Z)-isomer of any of the above compounds; and/or a pharmaceutically acceptable salt of any of the above compounds.

In some embodiments, Compound II is the (E) isomer of (R)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile or a pharmaceutically acceptable salt thereof.

In some embodiments, Compound II is the (Z) isomer of (R)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile or a pharmaceutically acceptable salt thereof.

In some embodiments, Compound II is a mixture of (E) and (Z) isomers of (R)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile or a pharmaceutically acceptable salt thereof.

### EXAMPLES

### Example 1. Methods

### A. Subjects

Patients presenting with thrombosis and thrombocytopenia, occurring after vaccination with the AstraZeneca vaccine AZD1222 (formerly ChAdox1 nCoV-19), were recruited.

### B. Antibodies and Reagents

Mouse monoclonal IgG2b antibody against human CD32 (IV.3) was purified from hybridoma cells supernatant, and IV.3 F(ab) fragment was made using Pierce Fab Preparation kit (Thermo Fisher Scientific, Catalog #44985).

Serum Preparation: Patient and healthy donor serum was collected following centrifugation (2000×g, 10 minutes, room temperature (RT)) of clotted whole blood. Patient sera was collected before and after treatment with dexamethasone and intravenous immunoglobulin (IVIg; see Table 1 in Example 2).

### C. Human Platelet Preparation

Washed platelets were prepared from citrated whole blood as described in Nicolson et al., Low-dose BTK inhibitors selectively block platelet activation by CLEC-2, Haematologica 106(1):208-19 (2021). Briefly; citrated blood was taken from healthy, drug-free volunteers and mixed (1:10, v/v) with acid citrate dextrose and centrifuged (200×g, 20 minutes, RT) to produce platelet rich plasma. Platelet rich plasma was then centrifuged (1000×g, 10 minutes, RT) in the presence of 0.2 μg/mL prostacyclin. The platelet pellet was resuspended in modified-Tyrode's-HEPES buffer, prepared as described in Nicolson et al., acid citrate dextrose and 0.2 μg/mL prostacyclin and centrifuged (1000×g, 10 minutes, RT). Platelet pellet was resuspended in modified-Tyrode's-HEPES buffer to a concentration of 2×10⁸/mL and allowed to rest for 30 minutes prior to use.

### D. Light Transmission Aggregometry (LTA)

Aggregation was measured in washed platelets (2×10⁸/mL) under stirring conditions (1200 rpm) at 37° C. using a light transmission aggregometer (Model 700, ChronoLog) for 20 minutes following stimulation with serum (1:15, v/v). Washed platelets were pre-incubated with IV.3 F(ab) for 5 minutes or with inhibitors for 10 minutes prior to stimulation with serum. Modified-Tyrode's-HEPES buffer or dimethyl sulfoxide (DMSO) was used as vehicle.

### E. Statistical Analysis

All data are presented as mean±standard error of the mean (SEM), p<0.05 was considered statistically significant. Statistical analysis was performed in GraphPad Prism 9 (GraphPad Software Inc.) using one or two-way ANOVA with Dunnett corrections for multiple comparisons.

### Example 2. Results

A. Patients with Vaccine-Induced Thrombosis and Thrombocytopenia Syndrome (VITT) Demonstrate Thrombosis, Thrombocytopenia, Elevated D-Dimer Levels and Anti-Platelet Factor 4 (PF4) Antibodies

The presentation, investigation results, treatment and outcomes of seven patients with VITT are summarized in Table 1.

TABLE 1
Summary of patients with VITT.

| | Patient 1 | Patient 2 | Patient 3 | Patient 4 | Patient 5 | Patient 6 | Patient 7 |
|---|---|---|---|---|---|---|---|
| Age | 48 | 32 | 21 | 46 | 43 | 44 | 42 |
| Sex | Male | Female | Male | Female | Female | Male | Male |
| Platelet count at | 16 | 98 | 113 | 7 | 11 | 35 | 21 |
| presentation (×10⁹/L) | | | | | | | |
| normal range 150-450 | | | | | | | |
| D dimer (ng/ml) | 62342 | 6574 | 22903 | 31301 | 30324 | 6807 | 27000 |
| normal range 0-250 | | | | | | | |
| Fibrinogen (g/L) | 1.2 | <0.35 | 0.98 | 1.1 | 1.01 | <0.35 | 2.36 |
| normal range 1.5-4 | | | | | | | |
| Prothrombin Time | 1.2 | 1.5 | 1.3 | 1.2 | 1.1 | 1.4 | 1.1 |
| Ratio | | | | | | | |
| normal range 0.8-1.2 | | | | | | | |
| Activated Partial | 1 | 1.7 | 0.8 | 1.1 | 1.2 | 1.6 | 1.3 |
| Thromboplastin Time | | | | | | | |
| Ratio | | | | | | | |
| normal range 0.8-1.2 | | | | | | | |
| HIT Antibody Screen | 2.45 | 2.17 | 2.8 | >3.0 | 1.77 | 2.6 | >3.0 |
| (Optical density) | | | | | | | |
| normal range 0.01-0.4 | | | | | | | |

Heparin-induced platelet activation (HITAlert) at presentation

| | | | | | | | |
|---|---|---|---|---|---|---|---|
| Platelet activation with | 24.79% | 31.2% | 55% | N/A | N/A | N/A | 75.31% |
| serum normal range ≤ | | | | | | | |
| 8% | | | | | | | |
| Platelet activation with | 18.53% | 18% | 36.5% | N/A | N/A | N/A | 22.63% |
| serum and heparin | | | | | | | |
| normal range ≤ 8% | | | | | | | |
| Platelet activation with | 0.64% | 3.68% | 1.43% | N/A | N/A | N/A | 4.38% |
| serum and excess | | | | | | | |
| heparin normal range ≤ | | | | | | | |
| 8% | | | | | | | |
| Clinical | CVST | CVST | Ischaemic | CVST | CVST | CVST | CVST |
| | | | stroke | | | | |
| Number of days post | 14 | 12 | 10 | 14 | 11 | 9 | 12 |
| vaccine at presentation | | | | | | | |
| Presentation symptoms | Headaches; | Occipital | Headache | Headache | Headache, | Headache | Headache for |
| | Haematuria; | headache | for 2-3 | | aura, | and | 1 wk, |
| | Petechial | | days; | | petechial | vomiting for | development |
| | rash; | | Collapse; | | rash | a few hours, | of rightside |
| | Subsequent | | Expressive | | | followed by | weakness; |
| | development | | dysphasia; | | | reduced | subsequent |
| | of left sided | | | | | conscious | seizure and |
| | weakness | | | | | level | collapse |
| Co-morbidities | Prostatitis | None | None | Hypothyroidism; | None | None | None |
| | | | | Fibromyalgia | | | |
| | | | | Anxiety | | | |
| Medications | None | None | None | Levothyroxine; | None | None | None |
| | | | | Sertraline; | | | |
| | | | | Amitriptyline | | | |
| Imaging findings at | CVST | CVST | Acute left | CVST | CVST | CVST; left- | CVST; |
| presentation | Subarachnoid | Subarachnoid | ICA | intraparenchymal | | side | subarachnoid |
| | haemorrhage | haemorrhage | thrombus | haemorrhage | | intracerebral | and |
| | | intraparenchymal | with | | | hemorrhage; | intraparenchymal |
| | | haemorrhage | multiple left | | | midline shift | hemorrhage; |
| | | | middle | | | | globalized |
| | | | MCA | | | | brain atrophy |
| | | | territory | | | | |
| | | | infarctions | | | | |
| Immunosuppression | IVIg 0.5 g/kg | IVIg 1 g/kg | IVIg 1 g/kg | IVIg 1 g/kg | IVIg 1 | IVIg 1 g/kg | IVIg 1 g/kg |
| regime used | OD for 2 | for 2 | single dose | single dose | g/kg on 2 | on 2 | single dose; |
| | consecutive | consecutive | Dexamethasone | Dexamethasone | nonconsecutive | nonconsecutive | dexamethasone |
| | days | days | 40 mg | 40 mg OD | days; | days | 40 mg (2 doses) |
| | Dexamethasone | Dexamethasone | OD for 4 | for 4 days. | dexamethasone | dexamethasone | |
| | 20 mg OD | 40 mg OD | days | | 40 mg OD | 40 mg | |
| | for 3 days | for 4 days | | | for 3 d | OD for 4 d | |
| Anticoagulant/ | Argatroban | Argatroban | Fondaparinux | Fondaparinux | Fondaparinux | Argatroban | None |
| Antiplatelet regime | Fondaparinux | | 7.5 mg OD | 2.5 mg SC | 7.5 | Fondaparinux | |
| used | 7.5 mg SC OD | | Apixaban | OD | mg SC | 7.5 mg SC | |
| | (when | | 5 mg BD (on | (whilst | OD | OD (when | |
| | platelets | | discharge) | platelets <50 × | | platelets | |
| | normalized) | | | 10⁹/L) | | $050 3109/ | |
| | | | | Fondaparinux | | L) | |
| | | | | 7.5 mg SC | | | |
| | | | | OD (when | | | |
| | | | | platelets ≥50 × | | | |
| | | | | 10⁹/L) | | | |
| | | | | Dabigatran | | | |
| | | | | 150 mg BD | | | |
| | | | | (on | | | |
| | | | | discharge) | | | |
| Other Treatments | Intubation | Intubation, | Thrombectomy | None | Plasma | Intubation, | Intubation, |
| Required | | thrombectomy | | | exchange | thrombectomy, | mannitol |
| | | | | | | decompressive | |
| | | | | | | craniotomy, | |
| | | | | | | plasma | |
| | | | | | | exchange, | |
| | | | | | | platelet | |
| | | | | | | transfusion | |
| Timing of first serum | Post-IVIg and | Post-single | Pre- | Pre-treatment | After IVIg | After IVIg | Pre-treatment |
| sample | Dexamethasone | dose of | treatment | | and | and | |
| | | Dexamethasone | | | dexamethasone | dexamethasone | |
| Timing of second serum | N/A | Post -IVIg | Post-IVIg | Post-IVIg | Post-PEX | Post-PEX | N/A |
| sample | | and | and | and | | | |
| | | Dexamethasone | Dexamethasone | Dexamethasone | | | |
| Days post IVIg that | N/A - | 2 days | N/A - | 3 days | 4 days | 1 day | N/A; died < |
| platelet count rose | Nadir 59 × | | nadir 52 × | | | | 24 h after |
| >50 x109/L | 10⁹/L | | 10⁹/L | | | | IVIg |
| | (platelets 100 × | | (Platelets | | | | |
| | 10⁹/L 48 | | 198 × 10⁹/L | | | | |
| | hours after | | three days | | | | |
| | first IVIg | | after IVIg | | | | |
| | infusion) | | infusion) | | | | |
| Outcome | Clinically | Death | Clinically | Clinically | Clinically | 36-d | Death 24 h |
| | recovered at | (support | recovered at | recovered at | recovered | intensive | after |
| | time of | withdrawn | time of | time of | at time of | care | admission |
| | discharge | following | discharge | discharge | discharge | admission; | (rapid |
| | from hospital | confirmation | from | from hospital | from | ventilator- | development |
| | after 26 day | of brainstem | hospital | after a 16 day | hospital | associated | of global |
| | admission | death) | after a 10 | admission; 2 | after a 12-d | pneumonia; | ischemia |
| | | | day | further | admission. | Limited | before |
| | | | admission; | admissions | | neurological | thrombectomy |
| | | | ongoing | with | | recovery; | could be |
| | | | mild right | headaches | | remains in | performed). |
| | | | hand | and | | hospital. | |
| | | | weakness | drops in | | | |
| | | | and | platelet | | | |
| | | | expressive | counts; no | | | |
| | | | dysphasia. | further | | | |
| | | | | CVST; | | | |
| | | | | treated with | | | |
| | | | | (1st | | | |
| | | | | admission) | | | |
| | | | | prednisolone | | | |
| | | | | then | | | |
| | | | | (2nd | | | |
| | | | | admission) | | | |
| | | | | IVIg | | | |
| | | | | and | | | |
| | | | | rituximab; | | | |
| | | | | Remains in | | | |
| | | | | hospital. | | | |

BD, twice daily;
Continuous positive airway pressure, CPAP;
CVST, Cerebral venous sinus thrombosis;
ICA, Internal carotid artery;
IVIg, intravenous immunoglobulin;
MCA, Middle cerebral artery;
OD, once daily;
SC, Subcutaneous

All seven patients were Caucasian, under the age of 50, and had not previously had symptomatic COVID-19. Patients presented with thrombosis (6 patients with cerebral venous sinus thrombosis [CVST] and 1 patient with ischemic stroke) and thrombocytopenia 9 to 14 days after the first AZD1222 vaccination. All patients presented with headaches, and one also had expressive dysphasia, 10-14 days following dosing with AZD1222. At presentation, clinical investigation revealed that all patients were thrombocytopenic (range: 7-113×10⁹ platelets/L), with massively elevated D-dimer and low fibrinogen levels. Four patients were male, and three patients were female.

Despite no prior exposure to heparin, heparin-induced thrombocytopenia (HIT) screening with the anti-platelet factor 4 (PF4) IgG assay (Immucor catalog #HAT45G) showed strong reactivity in all patients. Using a Heparin Induced Platelet Activation (HIPA) assay (HITAlert™ kit; IQProducts Catalog IQP-396), sera in four of the patients tested showed platelet activation compared to patient serum that was reduced by low, and blocked by high, concentrations of heparin. These results are similar to reports of other patients with VITT. See, e.g., Greinacher et al., Thrombotic thrombocytopenia after ChAdOx1 nCov-19 vaccination, N Engl J Med, doi:10.1056/NEJMoa2104840 (2021); Schlutz et al., Thrombosis and thrombocytopenia after ChAdox1 nCoV-19 vaccination, N Engl J Med, doi:10.1056/NEJMoa2104882 (2021). Cross-sectional brain imaging verified the presence of cerebral venous sinus thrombosis (CVST) and intracerebral haemorrhage in three patients, and ischemic stroke caused by internal carotid artery thrombus in one patient.

All patients received intravenous immunoglobulin (IVIg) and the steroid dexamethasone, which is recommended by the British Society of Haematology guidelines for VITT (British Society for Haematology, Guidance produced from the expert haematology panel (EHP) focused on Covid-19 vaccine induced thrombosis and thrombocytopenia (VITT), b-s-h.org.uk/media/19530/guidance-version-13-on-mngmt-of-thrombosis-with-thrombocytopenia-occurring-after-c-19-vaccine_20210407.pdf (2021). Platelet counts improved over 1 to 4 days in all patients except one, who died 24 hours after presentation. At the time of writing, three patients had recovered and been discharged from the hospital with ongoing normal platelet counts, one patient remained in hospital, and two patients had died because of the sequelae of CVST and secondary intracerebral hemorrhage. One discharged patient who was taking dabigatran, relapsed with thrombocytopenia and headaches but without thrombosis or raised D-dimer, 8 weeks after discharge and required repeat treatment with IVIg and corticosteroids. Two patients received plasma exchange. Of note, IVIg has also been shown to rapidly inhibit HIT antibody induced platelet activation. Warkentin, High-dose intravenous immunoglobulin for the treatment and prevention of heparin-induced thrombocytopenia: a review, Expert Rev Hematol 12(8):685-98, doi:10.1080/17474086.2019.1636645 (2019). Patients also received non-heparin anticoagulation, and two patients required intensive care unit support.

B. Serum from Patients with VITT Induces Platelet Aggregation

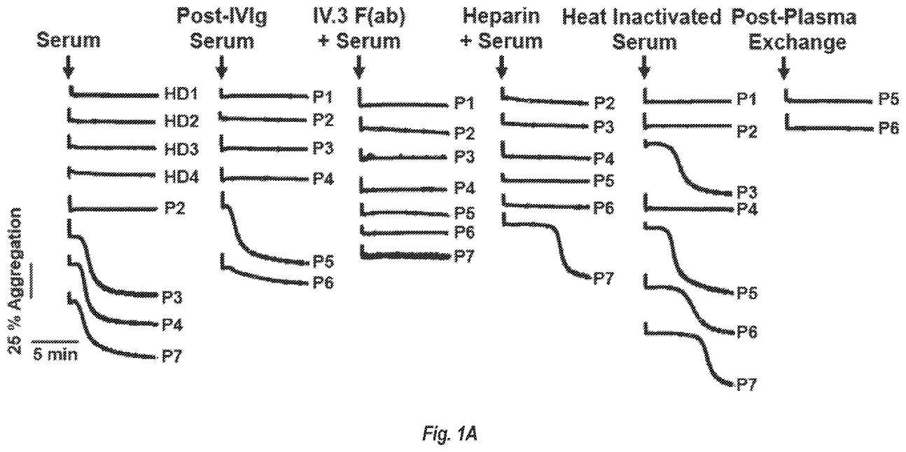

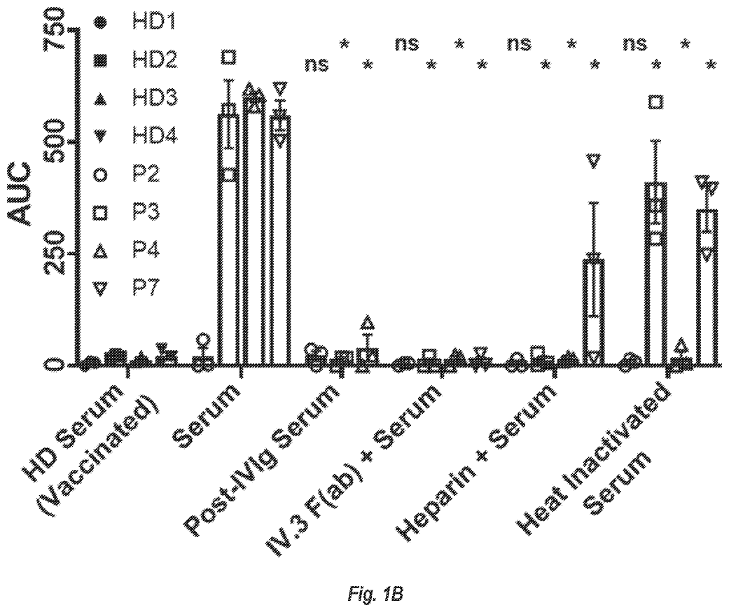

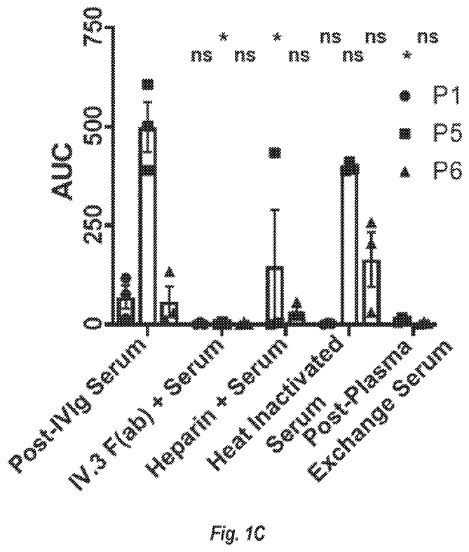

Serum was collected from healthy donors and patients with VITT. Patient 1 had serum collected after IVIg had been administered. Patients 2, 3 and 4 had serum collected both before and after IVIg administration. Patient 2 had received dexamethasone prior to their first serum collection. To investigate the effect on platelet activation, these sera were added to washed platelets and platelet aggregation was measured (FIGS. 1A-1C). Serum from patients with VITT triggered platelet aggregation to variable degrees depending on the platelet donor, which was abolished in post-IVIg treatment sera. Low-titer anti-PF4 antibodies have been shown to develop after vaccination in a small percentage of healthy individuals; however, they do not cause platelet activation. Aggregation was blocked after IVIg treatment, except in the 2 patients who did not clinically respond to IVIg and required plasma exchange (Figure TA). In these 2 patients, aggregation responses were blocked after plasma exchange. Eptifibatide treatment confirmed that responses were aggregation not agglutination. Three repeats were performed for each serum sample on platelets from healthy donors known to respond.

Addition of the integrin αIIbβ3 inhibitor eptifibatide (9 μM) inhibited the response to patient sera, confirming that this was aggregation rather than agglutination (data not shown).

C. Platelet Aggregation to Serum from Patients with VITT is Abolished by FCγRIIA Blockade and Heat Inactivation

Platelet activation in HIT is caused by antibody mediated clustering of FcγRIIA. Greinacher et al., Autoimmune heparin-induced thrombocytopenia, J Thromb Haemost 15(11):2099-114 (2017). To determine if a similar mechanism was involved in VITT we used an anti-FcγRIIA blocking IV.3 F(ab). Platelet activation by patient sera was abolished in the presence of IV.3 F(ab) demonstrating platelet activation in VITT is mediated via FcγRIIA (FIG. 1A).

The effects of PF4 and heparin in HIT in conjunction with patient sera were evaluated. Both PF4 and low concentrations of heparin have been shown to enhance platelet responses in HIT assays, whereas heparin at high concentrations inhibits any response. Rubino et al., A comparative study of platelet factor 4-enhanced platelet activation assays for the diagnosis of heparin-induced thrombocytopenia, H Thrombo Haemost 19(4):1096-102 (2021); Vayne et al., Beneficial effect of exogenous platelet factor 4 for detecting pathogenic heparin-induced thrombocytopenia antibodies, Br J Haematol 179(5):811-9 (2017); Padmanabhan et al., A novel PF4-dependent platelet activation assay identifies patients likely to have heparin-induced thrombocytopenia/thrombosis, Chest 150(3):506-15 (2016). No enhancement in the partial aggregation observed to patient 2 serum was observed in the presence of 10 μg/mL PF4 (data not shown). Low concentrations of heparin are known to enhance platelet responses in HIT assays, whereas high concentrations are inhibitory. In contrast, low (0.2 U/mL) concentrations of heparin prevented (5 of 7 patients) or delayed (2 of 7 patients) aggregation. High heparin concentration (100 U/mL) blocked aggregation (FIGS. 1B-1C).

Immune complexes that activate platelets via FcγRIIA have been reported in patients critically ill with COVID-19. In these patients, who had been exposed to heparin and displayed thrombocytopenia and thrombosis, HIT was ruled out, because of the lack of anti-PF4 antibodies and platelet activation independent of heparin. Platelet activation by these immune complexes was blocked by both low and high concentrations of heparin. We observed that heparin blocks platelet aggregation, which implies that the decision to withhold heparin use in patients with VITT perhaps should be revisited. Unfractionated heparin treatment has been reported in 1 patient with VITT without deleterious effect.

Anti-SARS-CoV-2 spike protein IgG antibodies from patients with severe COVID-19 have been shown to induce apoptosis and increase phosphatidylserine externalization in platelets mediated by FcγRIIA, although IgG aggregates or immune complexes could not be isolated from patient sera. It is possible that a similar mechanism is occurring in patients with VITT. Activation of FCγRIIA could give rise to phosphatidylserine exposure and procoagulant platelets, which may lead to the extensive thrombosis and thrombocytopenia observed in patients with VITT. To exclude platelet activation from other sources (such as thrombin and complement) in sera, heat inactivation (56° C., 45 minutes) of the three patient sera that caused activation was used. Warkentin et al., The platelet serotonin-release assay, Am J Hematol 90(6):564-72, doi:10.1002/ajh.24006 (2015). Heat inactivation of patient sera blocked aggregation in 3 of 7 patients (FIG. 1A), whereas minor effects on aggregation were observed with compstatin (a C3a inhibitor) and FUT-175 (a C3, C4, and C5 inhibitor; data not shown). These findings indicate that, although complement is not critical, it may reinforce platelet activation. Eculizumab (anti-C5 monoclonal antibody) treatment has been reported in 2 patients with VITT, in whom anticoagulation and IVIg or plasma exchange failed. Both patients rapidly improved. The involvement of complement, which mediates a broad range of thromboinflammatory reactions involving endothelium, monocytes, and neutrophils, as well as platelets, in VITT pathology should be considered. Normal serum complement levels in patients with VITT have been reported.

### D. Inhibition of Bruton's Tyrosine Kinase (Btk) by Rilzabrutinib Blocks Platelet Aggregation Induced by VITT Patient Serum

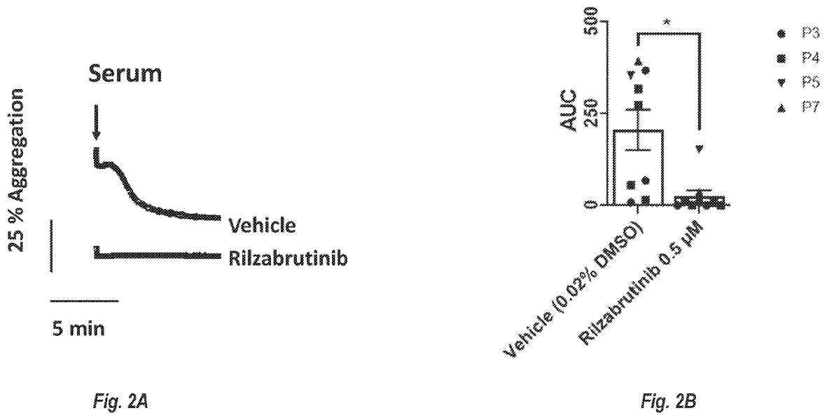

We tested if the Btk inhibitor rilzabrutinib (rilzabrutinib powder prepared in DMSO (final 0.02% DMSO) to achieve a concentration of 0.5 μM) could prevent platelet aggregation in patient sera. As shown in FIGS. 2A-2B, rilzabrutinib prevented platelet aggregation in patient sera (FIGS. 2A-B). Rilzabrutinib fully inhibited aggregation to 3 μg/mL collagen (results not shown).

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. A method for treating or preventing drug-induced thrombocytopenia (DITP) in a human subject in need thereof comprising administering to the human subject a therapeutically effective amount of at least one BTK inhibitor selected from:
Compound (I): 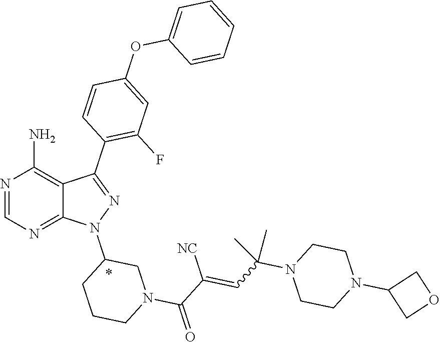 [SMILES: CC(C)(C=C(C#N)C(=O)N1CCCC(n2nc(-c3ccc(Oc4ccccc4)cc3F)c3c(N)ncnc32)C1)N1CCN(C2COC2)CC1]
where *C is a stereochemical center,
Compound (II): 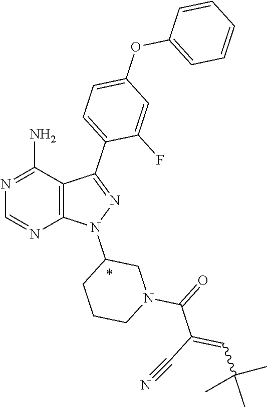 [SMILES: CC(C)(C)C=C(C#N)C(=O)N1CCCC(n2nc(-c3ccc(Oc4ccccc4)cc3F)c3c(N)ncnc32)C1]
N where *C is a stereochemical center, and pharmaceutically acceptable salts thereof.

2. A method for treating or preventing vaccine-induced thrombosis and thrombocytopenia syndrome (VITT) in a human subject in need thereof comprising administering to the human subject a therapeutically effective amount of at least one BTK inhibitor selected from Compound (I), Compound (II), and pharmaceutically acceptable salts thereof.

3. A method for increasing platelet count in a human subject with drug-induced thrombocytopenia (DITP) or vaccine-induced thrombosis and thrombocytopenia syndrome (VITT) comprising administering to the human subject a therapeutically effective amount of at least one BTK inhibitor selected from Compound (I), Compound (II), and pharmaceutically acceptable salts thereof.

4. A method for reducing platelet aggregation in a human subject with drug-induced thrombocytopenia (DITP) or vaccine-induced thrombosis and thrombocytopenia syndrome (VITT) comprising administering to the human subject a therapeutically effective amount of at least one BTK inhibitor selected from Compound (I), Compound (II), and pharmaceutically acceptable salts thereof.

5. The method of any one of claims 1-4, wherein, prior to administration, the human subject has at least one characteristic chosen from:
a. elevated D-dimer levels;
b. thrombosis; and
c. anti-platelet factor 4 (PF4) antibodies.

6. The method of any one of claims 1, and 3-5, wherein the drug-induced thrombocytopenia (DITP) is induced by administration of a small molecule, protein, or components, diluents, excipients or the like found in therapeutic treatments.

7. The method of any one of claims 1, and 3-6, wherein the drug-induced thrombocytopenia (DITP) is induced by administration of unfractionated heparin, enoxaparin, dalteparin, tinzaparin, acenocoumarol, acetaminophen, acetyldigoxin, alfacalcidol, allopurinol, alteplase, amphotericin B, argatroban, aspirin, atenolol, azathioprine, bivalirudin, bortezomib, capecitabine, captopril, carbamazepine, carboplatin, carfilzomib, ceftriaxone, cephalexin, chlorthalidone, cilastin/imipenem, clopidogrel, clozapine, cyclocytidine, dactinomucin/actinomycin, deferasirox, deferiprone, diflunisal, digoxin, dipyridamole, drospirenone/ethinylestradiol, eltrombopag, epoetin alfa, eporestenol, eptifibatide, famotidine, fluconazole, fluorouracil, furosemide, fusidic acid, ganciclovir, gemcitabine, gentamicin, glycoprotein IIB/IIA inhibitor, gold, hydrochlorothiazide, hydrochlorothiazide/triamterene, hydroxychloroquine, imatinib, inamrinone, intergrilin, ITP drugs, ixazomib, lenalidomide, levetiracetam, linezolid, melperone, menatetrenone, meropenem, methotrexate, metoprolol, molsidomine, nedaplatin, nicotinamide, nitrofurantoin, nonsteroidal anti-inflammatory drugs (NSAIDS) (e.g., ibuprofen, naproxen, celecoxib, diclofenac, and the like), octreotide, pantroprazole, penicillamine, phenytoin, piperacillin, propranolol, proton pump inhibitors, quinine, rifampin, ruxolitinib, ruxolitnib phosphate, sirolimus, spironolactone, streptokinase, sulfamethoxazole, sulfisoxazole, sunitinib, teicoplanin, temozolomide, temsirolimus, ticlopidine, tirofiban, trimethoprim/sulfamethoxazole, urokinase, valganciclovir, valproic acid, vancomycin, warfarin, or combinations thereof.

8. The method of any one of claims 1, and 3-6, wherein the drug-induced thrombocytopenia (DITP) is induced by administration of filgrastim (granulocyte colony stimulating factor; G-CSF), interferon, interferon alpha, peginterferon alfa 2B, peginterferon alfa 2B/ribavirin, factor VIII, TNF alpha, INF gamma, or combinations thereof.

9. The method of any one of claims 1, and 3-6, wherein the drug-induced thrombocytopenia (DITP) is induced by administration of abciximab, adalimumab, alemtuzumab, antibody-drug conjugates, anti-thymocyte globulin, brentuximab, cixutumumab, efaluzumab, natalizumab, rituximab, trastuzumab, or combinations thereof.

10. The method of any one of claims 2-5, wherein the vaccine-induced thrombosis and thrombocytopenia syndrome (VITT) is induced by administration of a vaccine.

11. The method of any one of claims 2-5, and 10, wherein the vaccine-induced thrombosis and thrombocytopenia syndrome (VITT) is induced by administration of a vaccine delivered in an adenoviral vector.

12. The method of claim 11, wherein the adenoviral vector comprises a therapeutic and/or prophylactic agent.

13. The method of claim 12, wherein the therapeutic or prophylactic agent is a gene therapy.

14. The method of any one of claims 10-13, wherein the vaccine is to prevent a coronavirus infection.

15. The method of claim 14, wherein the coronavirus infection is COVID-19.

16. The method of any one of claims 10-13, wherein the vaccine is to prevent an infection selected from measles, mumps, rubella, varicella, herpes simplex virus 1, herpes simplex virus 2, varicella, rotavirus, influenza, yellow fever, smallpox, hepatitis B, human papilloma virus, pneumococcus, hepatitis A, anthrax, diphtheria, acellular pertussis, hemophilus influenzae, including type B, meningococcus C, meningitis, typhoid, rabies, Lyme disease, tetanus, or any combination thereof.

17. The method of any one claims 1-16, wherein Compound I is (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, (S)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, a mixture of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and (S)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile; or an individual (E)- or (Z)-isomer of any of the above compounds; and/or a pharmaceutically acceptable salt of any of the above compounds.

18. The method of claim 17, wherein Compound I is the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt thereof.

19. The method of claim 17, wherein Compound I is the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt thereof.

20. The method of claim 17, wherein Compound I is a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt thereof.

21. The method of any one of claims 1-17, wherein Compound II is (R)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile, (S)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile, a mixture of (R)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile and (S)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile, or an individual (E)- or (Z)-isomer of any of the above compounds; and/or a pharmaceutically acceptable salt of any of the above compounds.

22. The method of claim 21, wherein Compound II is the (E) isomer of (R)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile or a pharmaceutically acceptable salt thereof.

23. The method of claim 21, wherein Compound II is the (Z) isomer of (R)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile or a pharmaceutically acceptable salt thereof.

24. The method of claim 21, wherein Compound II is a mixture of (E) and (Z) isomers of (R)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile or a pharmaceutically acceptable salt thereof.
